(19) United States
(12) Patent Application Publication
(10) Pub. No.: US 2018/0220877 A1
(43) Pub. Date: Aug. 9, 2018

(54) Hands-Free Arthroscopic Outflow Control Device

(71) Applicant: SeaChange Development, LLC, Boston, MA (US)

(72) Inventor: Michael Doyle Mason, Boston, MA (US)

(73) Assignee: SeaChange Development, LLC, Boston, MA (US)

(21) Appl. No.: 15/753,276

(22) PCT Filed: Jul. 15, 2016
(86) PCT No.: PCT/US16/42393

§ 371 Date: Feb. 17, 2018

Related U.S. Application Data
(60) Provisional application No. 62/194,053, filed on Jul. 17, 2015.

(51) Int. Cl. A61B 1/015; A61M 1/00

(52) U.S. Cl. CPC A61B 1/015; A61B 17/00234; A61M 2205/078; A61M 1/0035

(57) ABSTRACT

A hand-free arthroscopic outflow control device (100) having a body (10) featuring a multi port valve body comprising a sleeve (50) fitted with a piston (40) actuated by a pedal (30), the sleeve (50) communicating with a surgical site inflow port (52), a surgical theatre inflow port (54) and an outflow port (56) disposed between these respective inflow ports (52,54), so that the piston (40) can be positioned alternatively to vent the outflow port (56) under suction by drawing from one only of the surgical site inflow port (52) and the surgical theatre inflow port (54).

### TECHNICAL FIELD

The invention relates to arthroscopic surgery, and related surgical techniques that are characterised as minimally invasive—such as laparascopy, endoscopy, and similar procedures.

### BACKGROUND ART

Arthroscopy is a minimally invasive surgical technique performed on a joint using an arthroscope, which is an endoscope inserted into a joint through a small incision.

Typically, the arthroscope is placed through a cannula that has both an inflow and an outflow portal, each of which is provided with a simple stopcock arrangement for flow control. An arthroscopic camera provides a view of the surgical site via this cannula. A second incision provides access for a surgical implement to be introduced and used at the surgical site.

A suction canister downstream of the stopcock is used to evacuate irrigation fluid via the outflow portal, and the surgeon or an assistant evacuates the surgical site as required. During the surgery the stopcock is normally in the closed position until the surgeon needs to evacuate blood and surgical debris, which obscures vision of the surgical site. When evacuation is performed manually by the surgeon they must interrupt their work, or alternatively when using an assistant for this task issue verbal commands.

While this status quo permits fine control of visibility at the surgical site, there is also typically an overflow of excess surgical fluid from time to time, which collects in puddles on the floor of the surgical theatre around the patient and surgical team. Some attempts have been made to deal with pooled surgical fluids. At a minimum, the surgical team may use waterproof footwear, transparent gators and the like. Collection pouches around the surgical site, or absorbent mats may also be used. None of these measures are wholly satisfactory.

There is a need in light of prevailing equipment and practices for improved devices or methods that address these and other limitations pertaining to arthroscopic and related surgeries, or at least provide a useful alternative.

### SUMMARY OF INVENTION

The present invention arises from a recognition that fluid irrigation in minimally invasive surgical procedures can be managed by a hands-free device which permits a surgeon to operate without interruption to their surgical flow.

Arthroscopic surgeries, and arthroscopic knee surgeries in particular are favoured applications of the device.

There is provided, according to an aspect of the present invention, a hand-free arthroscopic outflow control device having a body featuring a multiport valve body comprising a sleeve fitted with a piston actuated by a pedal, the sleeve communicating with a surgical site inflow port, a surgical theatre inflow port and an outflow port disposed between these respective inflow ports, so that the piston can be positioned by the pedal alternatively to vent the outflow port under suction by drawing from one only of the surgical site inflow port and the surgical theatre inflow port.

A surgical site can be drained—hands-free, as required—by selectively depressing the foot-operated pedal. The device is operated under negative pressure (that is, suction) applied to the outflow port, which selectively drains fluid from one of the inflow posts but not both.

As the device is foot-operated, it is conveniently placed on the floor, and is preferably designed so the surgical theatre inflow port opens directly to an area on the floor of the surgical theatre where excess fluid pools.

As the outflow port is positioned between the inflow ports, the relative position of the piston in the valve body permits selection between one of two mutually exclusive flow paths: from only one of the surgical site inflow port and surgical theatre inflow port to the outflow port.

Cross-contamination from the surgical theatre to the surgical site is avoided as the piston blocks backflow from the surgical theatre to the surgical site as a consequence of the outflow port being located intermediate the surgical site inflow port and the surgical theatre inflow port. The inflow ports can be accordingly characterised as functioning independently or exclusively.

As will be appreciated, the device offers manifold advantages. Hands-free operation permits a surgeon to operate uninterrupted when a surgical site is to be evacuated, and promotes ‘surgical flow’. Clearance of excess fluids from a floor of a surgical theatre is also conveniently achieved, which avoids potential slip hazards. Advantageously, the device can be simply incorporated in-line with irrigation tubing with existing surgical equipment, and no disruptive change to existing surgical procedures and protocols.

### BRIEF DESCRIPTION OF DRAWINGS

FIG. 1 is a perspective view of a hands-free arthroscopic outflow control device according to an embodiment of the present invention.

FIG. 2 is a further perspective view of the device of FIG. 1, depicted from its underside.

FIGS. 3 and 4 are views of the device of FIG. 1, depicted in plan from above and below.

FIGS. 5 and 6 are elevations of the device of FIG. 1, from the toe end and heel end respectively.

FIG. 7 is a cross-sectional view corresponding to FIG. 6.

FIG. 8 is a side elevation of the device of FIG. 1, and FIG. 9 is a cross-sectional view corresponding to FIG. 8.

FIGS. 10 and 11 are details of cross-sectional views taken from FIGS. 7 and 9 respectively.

FIGS. 12 to 19 depict view of a hands-free arthroscopic outflow control device according to an alternative embodiment to that depicted in connection with FIGS. 1 to 11.

FIGS. 12 and 13 are perspective views of the device of the alternative embodiment, corresponding to FIGS. 1 and 2.

FIG. 14 is a view of the embodiment of FIGS. 12 and 13 depicted in plan from above, while FIG. 15 is a view of the same embodiment in elevation.

FIGS. 16 and 17 are further elevation from the heel end and toe end respectively.

FIGS. 18 and 19 are cross-sectional views of the embodiment of FIGS. 12 and 13, similar to those of FIGS. 7 and 9.

FIGS. 20 and 21 and details isolating the valve arrangement as depicted in section in FIGS. 18 and 19.

FIG. 22 is an exploded view of components of the device of the alternative embodiment.

### DESCRIPTION OF EMBODIMENTS

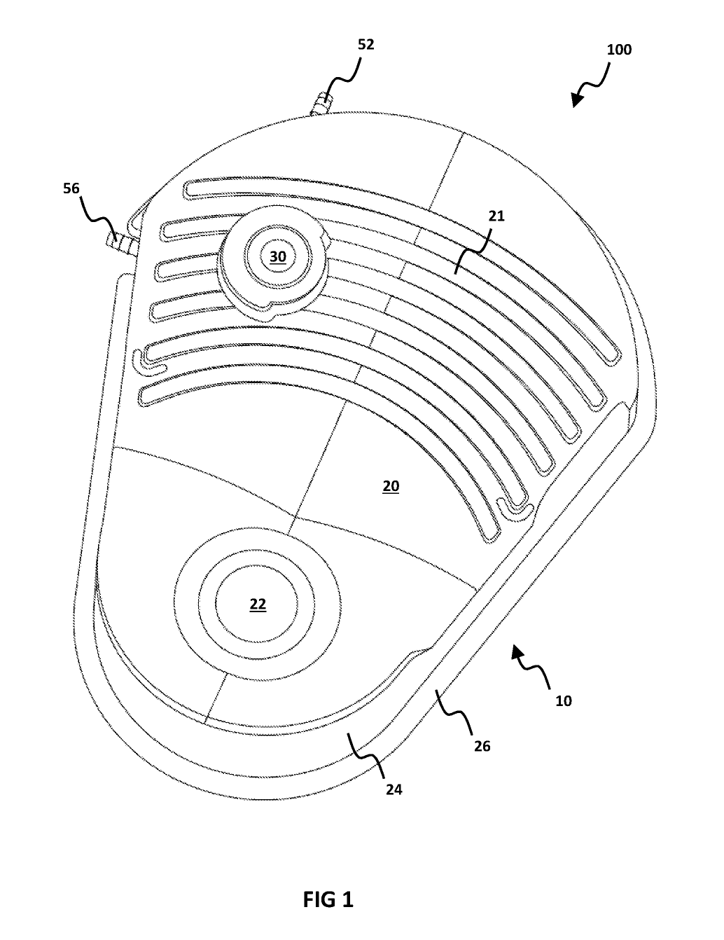

FIGS. 1 to 4 collectively depict different views of a device 100, according to an embodiment of the invention, for controlling flow of surgical fluid in a hands-free manner by a surgeon, such as for an arthroscopic surgery. The remaining views FIGS. 5 through to 11 are elevations or sections which complete the visual depiction of the device 100.

The device 100, as depicted, has a body 10 which presents on its upper side a platform 20 for a surgeon to rest his foot during surgery. The device 100 is in use placed on a floor of a surgical theatre, so that a lower or underside of the body 10 rests on the floor, placed near the surgeon, where surgical fluid may tend to pool.

The device 100 features a pedal 30, by which the device 100 is foot actuated by gently depressing the pedal 30 with the foot as required to drain from the surgical site. The pedal 30 ordinarily projects a small distance above the platform 20. The pedal 30 is directly connected to (in fact, integral with) a piston 40, which is slidingly received in a sleeve 50 formed in the body 10.

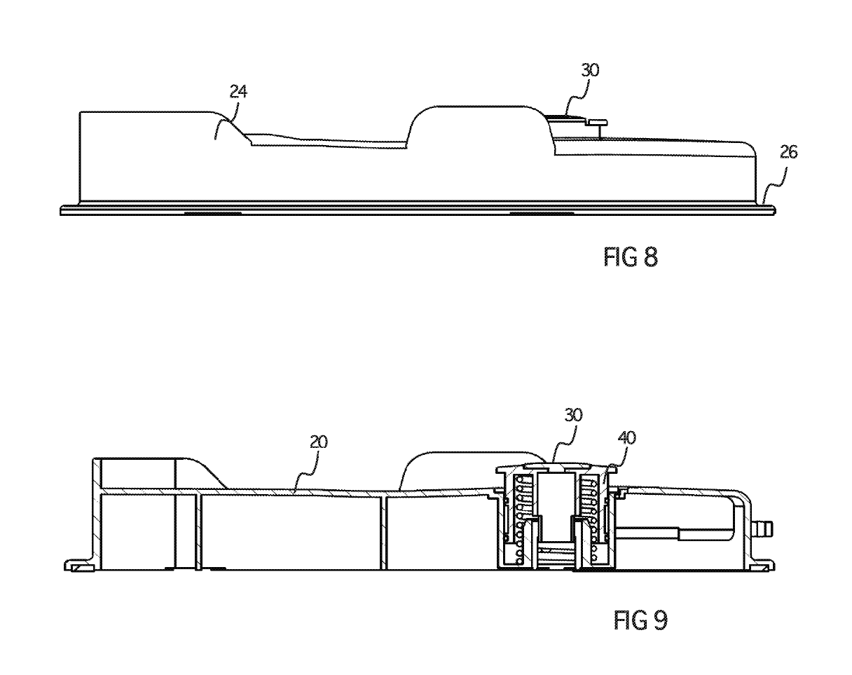

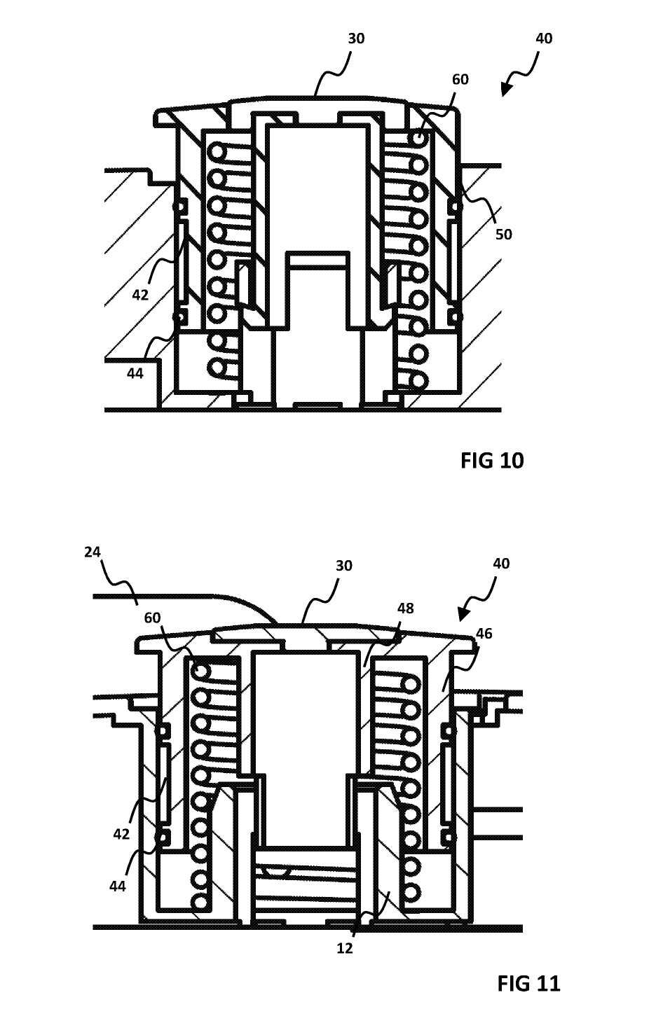

This is seen to best effect in the sectional details of FIGS. 10 and 11, though also in originating views of FIGS. 7 and 9. Furthermore, the piston 40 is retained in the sleeve 50 as described below—and so the piston 40 slides within the sleeve 50 within upper and lower bounds.

The platform 20 features non-slip ribs 21, and a heel pad 22. The ribs 21 are formed slightly proud of the platform 20, and act to drain any incidental fluid which may be on the soles of the surgeon's shoes, or which happens to splash on the device 100. The heel pad 22 may be non-slip, though principally serves as a visual or design cue.

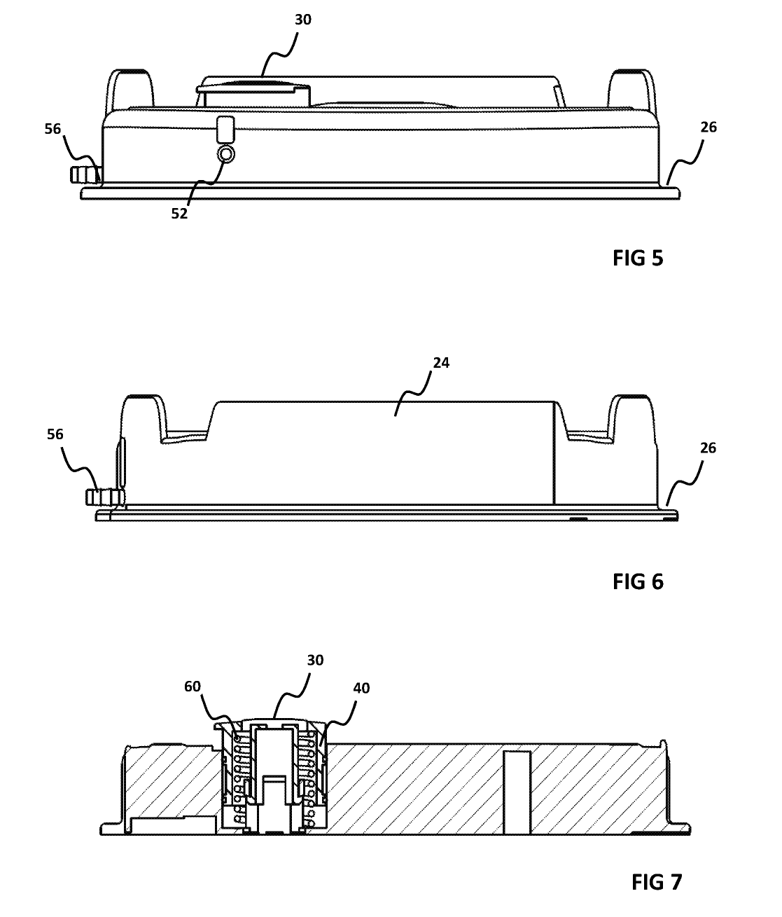

A sidewall 24 bounds the platform 20, and extends down to the floor, terminating in a stabilising footing 26 at floor level. The peripheral sidewall 24 in places extends beyond the level of the platform 20, as most clearly seen in the elevations of FIGS. 5, 6, and 8. This serves to locate the surgeon's foot, and avoid incidental activation of the pedal 30 during surgery.

As described, the pedal 30 ordinarily extends a small distance above the platform 20, under influence of the biasing action of a helical spring 60, which acts at its lower end acts against the body 10 and at its upper end against an underside of the pedal 30.

The piston 40 and sleeve 50, in conjunction with two inflow valves 52, 54, and an outflow valve 56, in effect co-operatively form a multivalve body. This pedal-actuated multivalve body permits hands-free operation by the surgeon, and achieves drainage alternatively from the floor of the surgical theatre or the surgical site, as required.

Upon hands-free actuation of the pedal 30, the piston 40 slides down in the sleeve 50, which results in the outflow valve 56 draining via surgical site inflow valve 52, rather than the surgical theatre inflow valve 54. The respective inflow valves 52, 54 commonly drain under suction through the outflow valve 56.

The surgical site inflow port 52 extends between the peripheral wall of the sleeve 50 towards its upper end, and terminates at the sidewall 24 of the body 10 in a barbed tube connector.

The surgical theatre inflow port 54 is formed at a base of the sleeve 50, namely its lower axial end, and operatively draws from the floor of the surgical theatre. As can be seen, this inflow port 54 is preferably surrounded by a number of discrete pads radially distributed around the base of the sleeve 50. The pads are in contact with the floor of the surgical theatre, thus presenting a number of spaced apart channels through which fluid can be drawn from the floor of the surgical theatre, and feed into the surgical theatre inflow port 54.

The outflow port 56—as with the surgical site inflow port 52—also extends between the peripheral wall of the sleeve 50 and a sidewall 24 of the body 10. The outflow port 56 is positioned below the surgical site inflow valve 52, as is apparent from the drawings. The outflow port 56 is closer to the lower end of the sleeve 30, and draws from the peripheral wall of the sleeve 50 between the surgical theatre inflow valve 52 and the surgical theatre inflow valve 54.

As is apparent from the drawings, both the surgical site inflow port 52 and the outflow port 54 terminate with a barbed tube fitting for removably attaching standard size surgical tubing.

When the piston 40 is ordinarily biased there is no fluid communication between the surgical site inflow port 54 and the outflow port 56. The surgical site inflow port 52 is in effect blocked, and does not drain. The surgical site inflow port 52 is blocked by the piston 40, in the region of the relief belt 42 of the piston. More particularly, the surgical site inflow port 52 is blocked by the upper and lower radial seals 44 that bound the belt 42 of the piston 40.

When the pedal 30 is depressed—by foot—the piston 40 slides downwardly in the sleeve 50 towards the lower extent of its range. The pedal 30 is depressed against the action of the helical spring 60. As the piston 40 travels towards the lower end of its range the surgical theatre inflow port 52 is blocked by the piston 40, and a fluid communication channel is instead established between the surgical site inflow port 52 and the outflow port 56. This fluid communication is effected via the relief belt 42, which provides a path from the surgical site inflow port 52 to the outflow port 56, via an annular void between the piston 40 and sleeve 50, as bounded by opposing ends of a radial belt 42.

Thus device 100 achieves selective porting by use of the relief belt 42, which as indicated is of reduced radial diameter extending, and extending axially between two radial seals 44. The radial seals 44 are provided in the form of O-rings seated in matching glands. Each of these spaced apart radial seals 44 bound opposite ends of the relief belt 42. As with other details concerning the multi-valve arrangement, this is seen to best effect in the details of FIGS. 10 and 11.

While various other design arrangements are possible, using a reduced diameter relief belt 42 on the piston 40 permits reliable and smooth axial alignment of the piston 40 in the sleeve 50. The use of this spaced apart pair of radial seals 44 is advantageous in ensuring that any fluid from the surgical site does not leak along the sleeve 50.

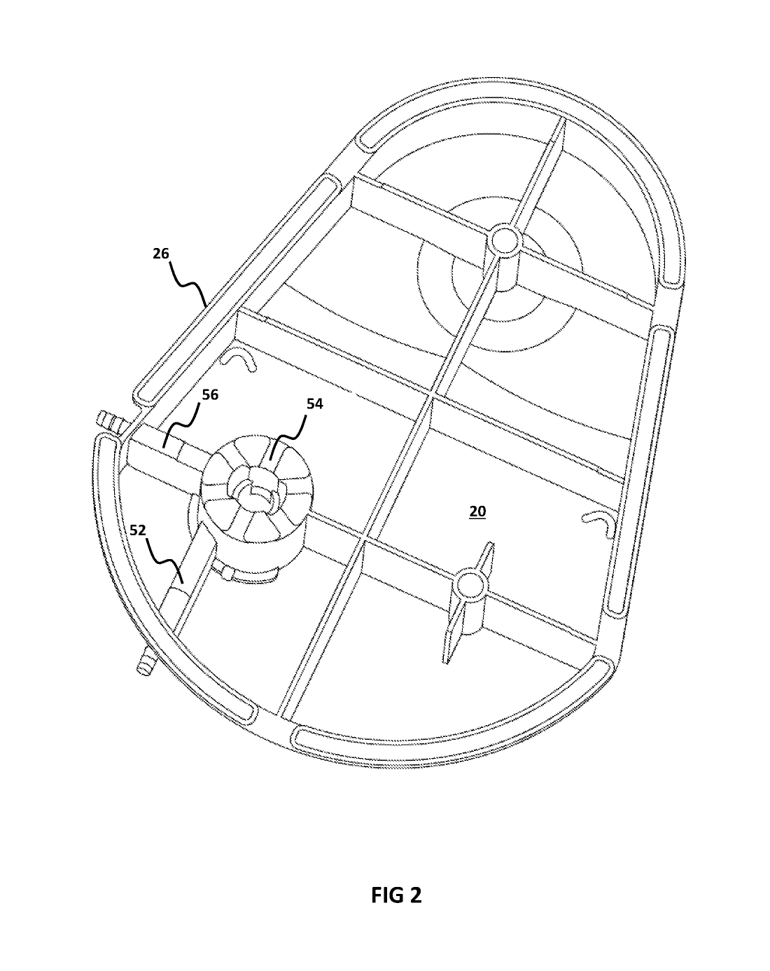

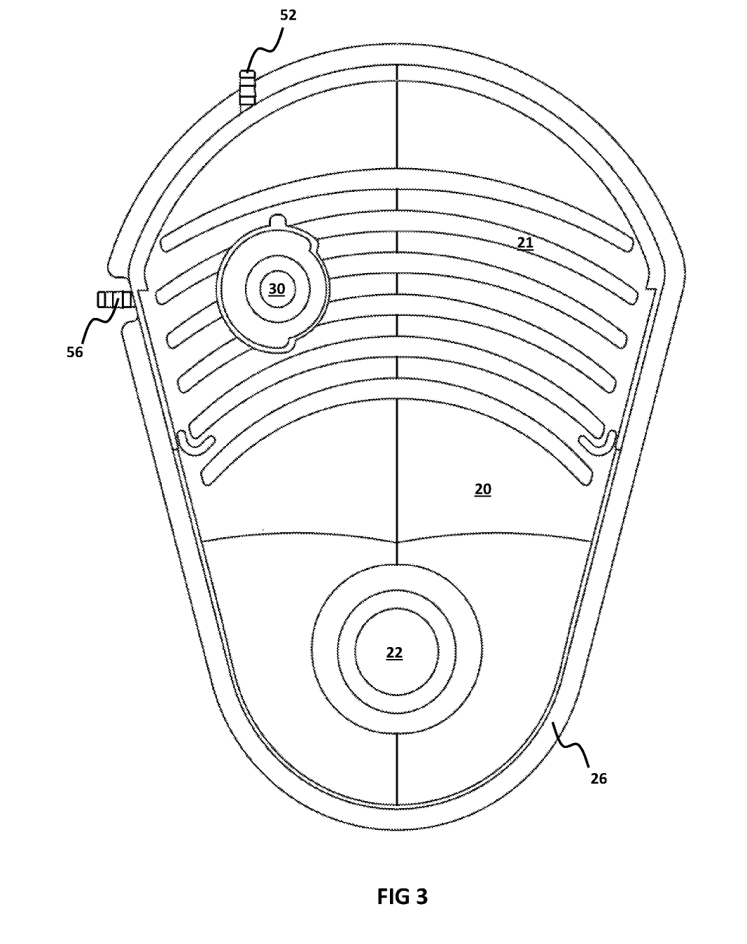

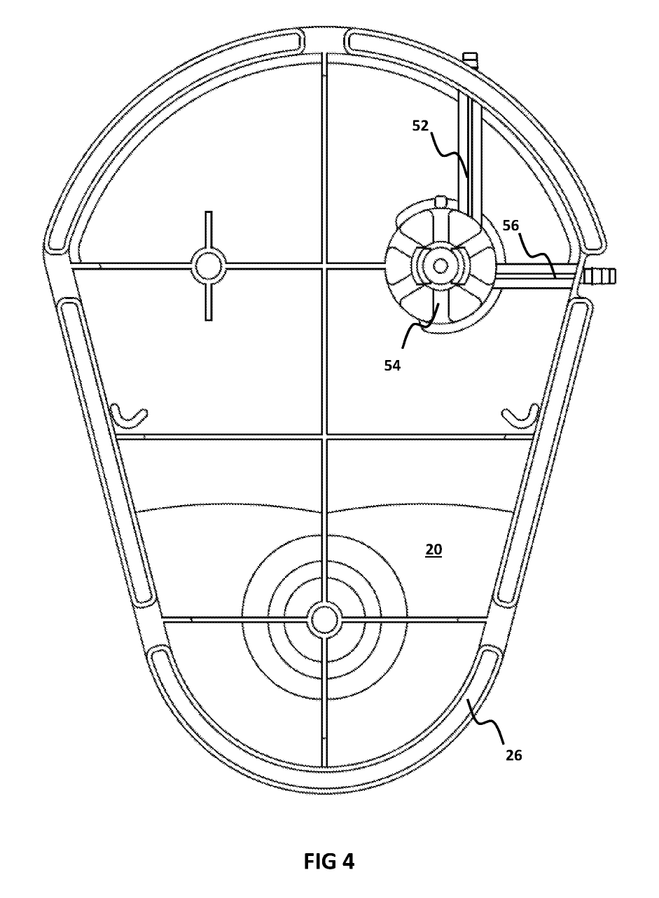

As is most clearly apparent in FIGS. 2 and 4, the underside of the body 10 is largely void. The underside does however include a lattice of structural webbing to support and reinforce the device 100.

The peripheral footing 26 which extends around the periphery of the sidewall 24 of the body 10 is interrupted at spaced apart intervals with cut away niches. These niches provide a small gap between the device 100 and the floor, which presents channels for ingress of fluid from the floor surrounding the device 100. These channels are low profile, but sufficient to allow ingress of excess surgical fluid which may have collected on the floor of the surgical theatre. Suction supplied via the outflow port 56 coupled with fluid surface tension is sufficient to drain surrounding fluid which may have collected about the device 100.

Also, as can be observed in the drawings (refer FIGS. 2 and 4), the peripheral footing 26 along its underside features recessed hollows to permit interference fitting of matching rubber strips, which serve to inhibit slipping of the device 100 on the floor of the surgical theatre.

The body 10 also features internal to the sleeve 50 an upwardly extending neck which is collared by the helical spring 60, which is fitted in snug fit—serving to both retain and centrally position and align the helical spring 60 within the sleeve 50.

The helical spring 60 engages the piston 40 between the skirt 46 and a downwardly extending throat 48, which also serves to position the helical spring 60. The throat 48 snaps locks into the neck, which serves to fit the piston 40 into the body, and more particularly to check the upper extent of the piston 40 under influence of the helical spring 60.

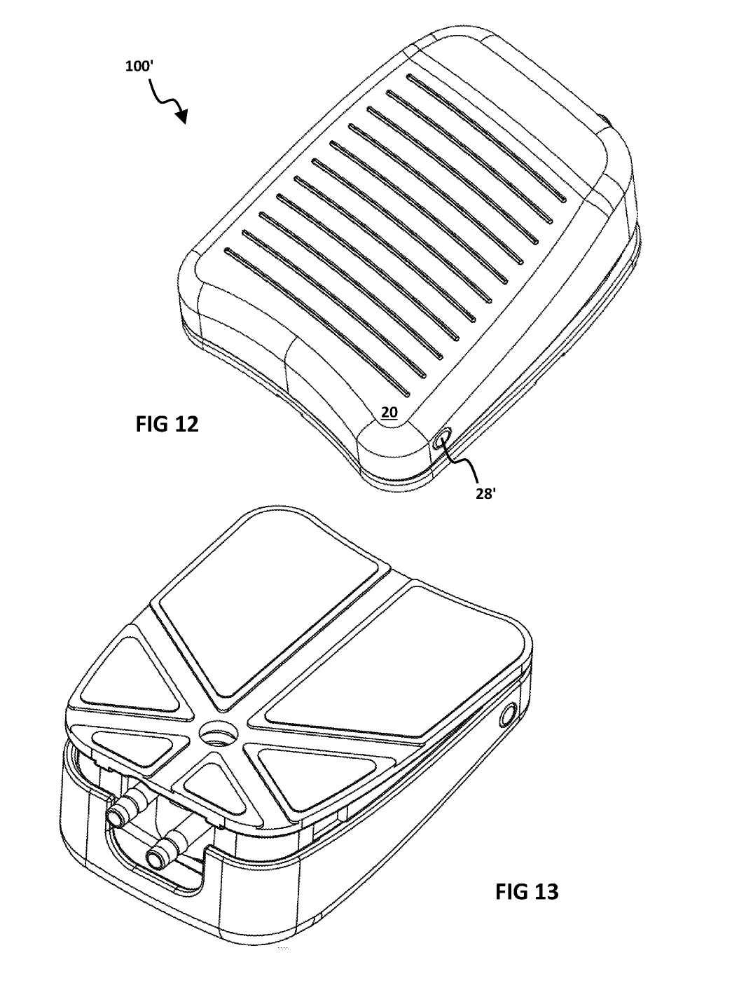

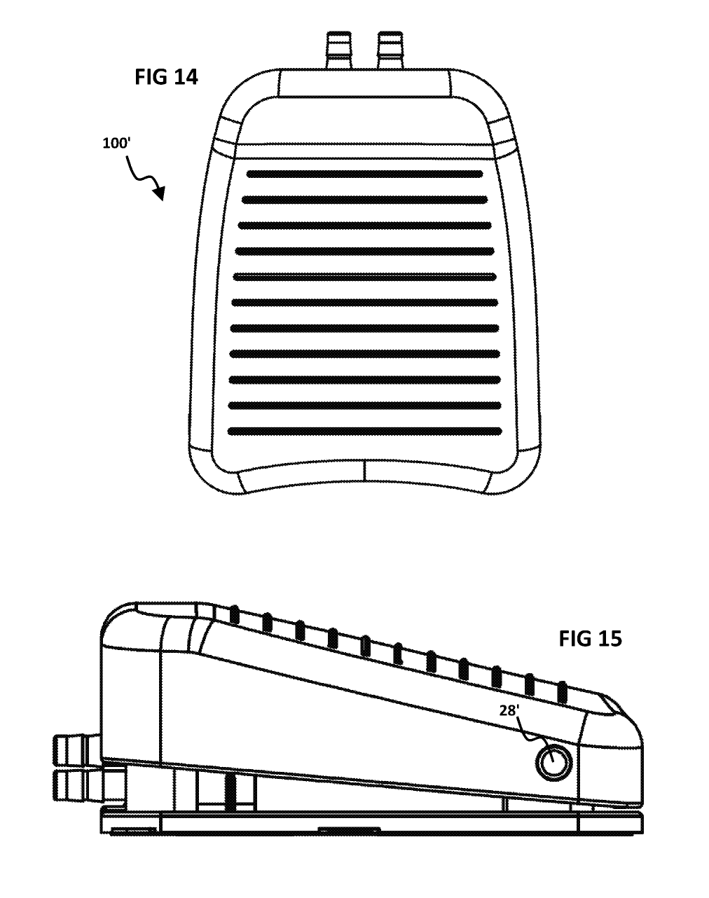

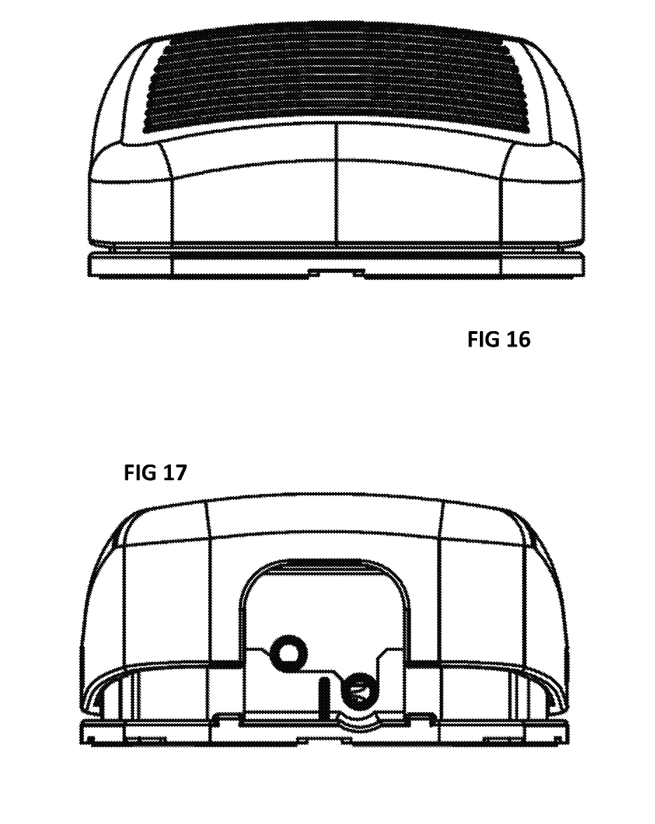

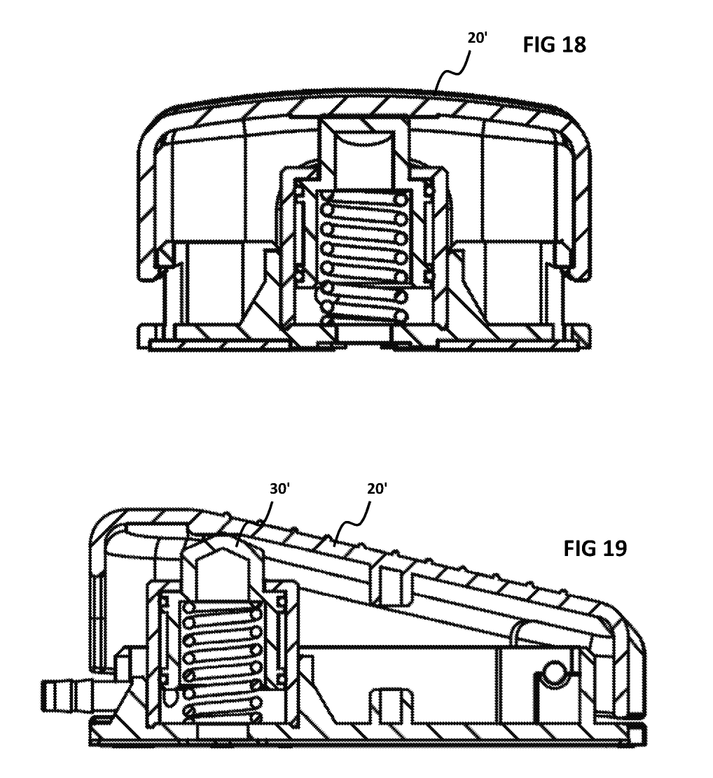

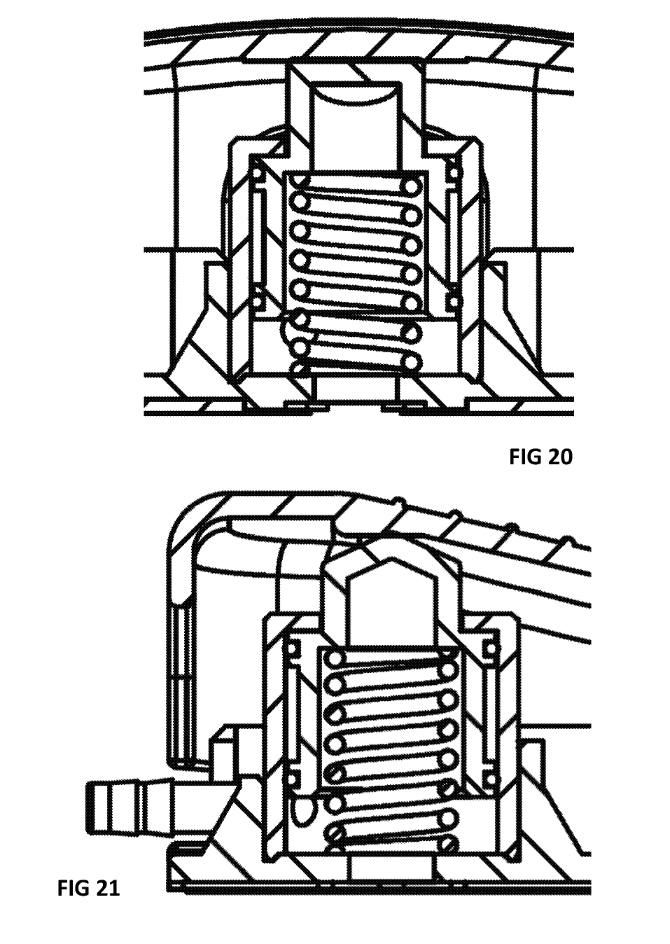

FIGS. 12 to 22 depict a device 100′ according to an alternative embodiment of the device 100 of FIGS. 1 to 11. While there are clear aesthetic and ergonomic differences between the devices 100, 100′—as is apparent—both embody the same operating principles, particularly as regarding the valving arrangement. Some construction details vary between embodiments of the device 100, 100′, as will be appreciated. Corresponding reference signs denote corresponding features.

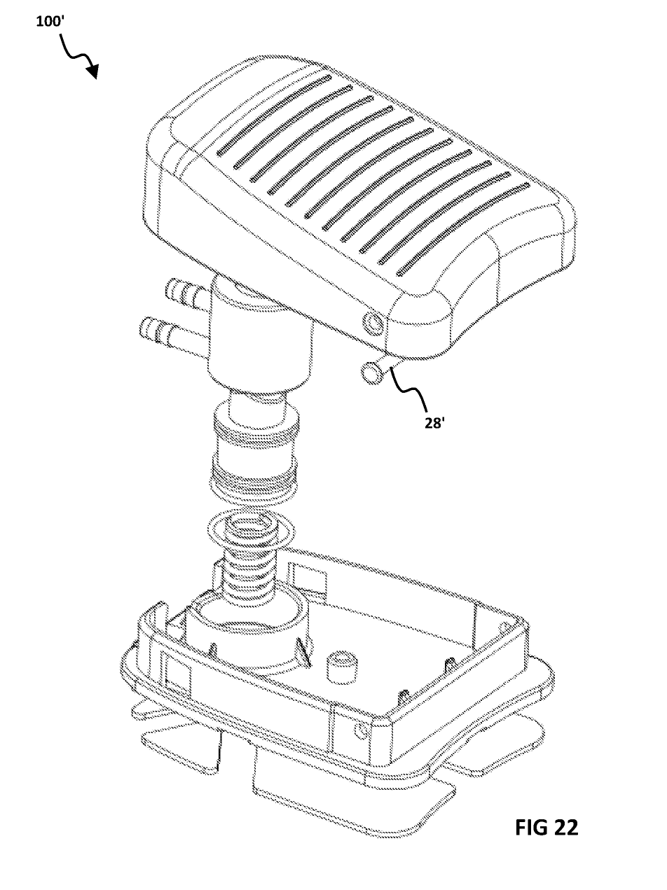

The main operative distinction between the devices 100, 100′ is that the device 100′ of FIGS. 12 to 22 is adapted for a surgeon to depress pedal 20′, which acts against a stem 30′ integral with piston 40′. Pedal 20′ is hinged at a toe-end of the device 100′ by a pivot pin 28′, seen to best advantage in FIG. 22. An underside of the pedal 20′ abuts the stem 30′, but is not attached to the platform 20′. This contrasts with device 100 as described above, in which a surgeon depresses pedal 30, which is directly connected with piston 40.

As will be appreciated, various other minor constructional details differ between embodiments of the device 100, 100′, and neither embodiment is limiting as further modifications and variations are embraced within the scope of the invention.

1. A hand-free arthroscopic outflow control device having a body featuring a multi port valve body comprising a sleeve fitted with a piston actuated by a pedal, the sleeve communicating with a surgical site inflow port, a surgical theatre inflow port and an outflow port disposed between these respective inflow ports, so that the piston can be positioned via operation of the pedal alternatively to vent the outflow port under suction by drawing from one only of the surgical site inflow port and the surgical theatre inflow port.

2. The device according to claim 1, further comprising a biasing element that biases the piston in a biased configuration in which the outflow port draws from the surgical theatre inflow port, and an actuated configuration in which the outflow port draws from the surgical site inflow port.

3. The device according to claim 2, wherein the piston has a relief belt bounded by spaced apart radial seals that serve to contain the surgical site inflow port in the biased configuration, and permit fluid communication between the surgical site inflow port and the outflow port in the actuated configuration.

4. The device according to claim 1, wherein the surgical site inflow port extends between a peripheral wall of the sleeve and a barbed tube fitting, and the outflow port also extends between the peripheral wall of the sleeve and a barbed tube fitting, the surgical site inflow port joining the sleeve above the outflow port.

5. The device according to claim 4, wherein the surgical theatre inflow port extends between a base of the sleeve and a floor upon which the device is operatively placed.

6. The device according to claim 2, wherein the pedal is configured to be depressed against biasing action of the biasing element in the form of a helical spring.

7. The device according to claim 6, wherein the pedal is integral with the piston.

8. The device according to claim 6, wherein the pedal is pivotally mounted to the body, and acts against a stem integral with the piston.
